(19) United States
(12) Patent Application Publication
(10) Pub. No.: US 2023/0131962 A1
(43) Pub. Date: Apr. 27, 2023

(54) OVARIAN RESERVE BIOMARKER AND USE THEREOF

(71) Applicant: CHA UNIVERSITY INDUSTRY-ACADEMIC COOPERATION FOUNDATION, Pocheon-si (KR)

(72) Inventors: Kyung-Ah LEE, Seoul (KR); Kyeoung-Hwa KIM, Seongnam-si (KR); Mi Kyoung KOONG, Seoul (KR)

(73) Assignee: CHA UNIVERSITY INDUSTRY-ACADEMIC COOPERATION FOUNDATION, Pocheon-si (KR)

(21) Appl. No.: 17/905,132

(22) PCT Filed: Aug. 26, 2020
(86) PCT No.: PCT/KR2020/011402

§ 371 Date: Nov. 30, 2022

(30) Foreign Application Priority Data
Feb. 28, 2020 (KR) 10-2020-0025534

(51) Int. Cl. C12Q 1/6883

(52) U.S. Cl. CPC C12Q 1/6883; C12Q 2600/118; C12Q 2600/158; C12Q 2600/178

(57) ABSTRACT

According to a composition and a kit for diagnosing ovarian reserve, and a method of providing information for diagnosing ovarian reserve by using the same, according to an aspect, the development of follicles may be predicted early. In addition, by predicting not only the number of oocytes but also the quality of oocytes, a comprehensive diagnosis of ovarian reserve may be made.

### TECHNICAL FIELD

The present disclosure relates to a biomarker composition and a kit for testing ovarian reserve, and a method of predicting ovarian reserve by using the same.

### BACKGROUND ART

The ovary is an organ that is responsible for two important functions: producing healthy oocytes, which are reproductive cells, and maintaining a normal reproductive cycle by secreting female hormones. A woman is born with a fixed number of primordial follicles at birth, and 20 to 30 primordial follicles resume growth every month during a fertile period, and only one follicle ovulates to produce a mature oocyte. In other words, a limited number of primordial follicles existing in the ovaries are continuously consumed, and as a woman ages, her fertility decreases rapidly, and thereafter, due to the depletion of primordial follicles, menopause is reached.

In modern society, the marriage age is gradually being delayed due to women's advancement into society, and accordingly, there is an issue of increasing age of mothers who give birth to their first child. In addition, there are various types of issues related to ovarian function, such as premature ovarian failure, in which reproductive cells are depleted before the age of 40, and loss of ovarian function due to radiation or chemicals for cancer treatment. The issues of ovarian function due to late marriage and increase in life expectancy are emerging as a social problem as it is linked to low birthrate due to infertility and a decrease in women's quality of life due to a decrease in female hormone secretion.

Ovarian reserve is evaluated to predict a woman's ovarian function and capacity to produce oocytes, but there is no absolute standard. It is usually impossible to monitor the number of primordial follicles in a woman's ovary or the development of early follicles with the naked eye. Therefore, whether or not follicles develop is indirectly determined by measuring the substances secreted during early follicle development. Currently, the most commonly used indicator of ovarian reserve is anti-Mullerian hormone (AMH). AMH is secreted from granulosa cells of early follicles (primary follicles or mature follicles) that have resumed growth and, due to its advantageous traits of fluctuating little according to the menstrual cycle and having high sensitivity, is used as a marker capable of providing knowledge about the development of follicles and the increasing number of granulosa cells. However, human AMH assay kits that have been developed and used to measure an amount of AMH have an issue of large analysis deviation, and there are limitations in that AMH alone cannot determine all ovarian functions and AMH cannot be used for qualitative evaluation of oocytes.

Accordingly, the present inventors overcame the above limitations by developing an ovarian reserve biomarker that predicts the function of an ovary at an early stage and predicts the quality of the oocytes as well as the function of the oocytes.

### DESCRIPTION OF EMBODIMENTS

### Technical Problem

An aspect provides a composition for diagnosing ovarian reserve, including an agent capable of measuring expression levels of miR-145-5p or miR-425-5p.

Another aspect provides a kit for diagnosing ovarian reserve, including an agent capable of measuring expression levels of miR-145-5p or miR-425-5p.

Still another aspect provides a method of detecting a marker to provide information for diagnosing ovarian reserve, including: measuring an expression level of miR-145-5p or miR-425-5p in a subject suspected of having ovarian hypofunction; and
comparing the measured expression level with an expression level of the gene of the normal control group.

### Solution to Problem

An aspect provides a composition for diagnosing ovarian reserve, including an agent capable of measuring expression levels of miR-145-5p or miR-425-5p.

Another aspect provides a composition for diagnosing ovarian hypofunction or early menopause, including an agent capable of measuring expression levels of miR-145-5p or miR-425-5p.

The miRNA may be isolated from a biological sample isolated from a subject suspected of ovarian hypofunction or a subject suspected of having low ovarian reserve. The subject may be a mammal, and may be a human, a dog, a cat, a rat, a mouse, a hamster, a rabbit, a horse, sheep, a cow, a goat or a pig. The subject may be of a female phenotype.

The biological sample may be a blood-derived sample or a cell-derived sample. The blood-derived sample may be serum, plasma, and peripheral blood mononuclear cells (PBMC) separated from blood. The PBMC may include T-cells, B-cells, natural killer (NK) cells, monocytes, macrophages, dendritic cells, or a combination thereof. Preferably, the blood-derived sample may be serum or plasma. The sample may include transcripts or proteins.

The term “miR” or “micro RNA” refers to 21 to 23 non-coding RNAs that post-transcriptionally regulate gene expression by promoting degradation of target RNAs or inhibiting the translation thereof. Mature sequences of the miRNAs used herein may be obtained from a miRNA database (http://www.mirbase.org). In general, microRNA is transcribed into a precursor of about 70 nt to 80 nt (nucleotide) in length with a hairpin structure called pre-miRNA, and then cut by a dicer, an RNAse III enzyme, to be generated in a mature form. MicroRNA cleaves a target gene or inhibits translation. by forming a ribonucleotide complex called miRNP, and forming a complementary bond at the target site. More than 30% of human miRNAs exist in clusters, and after a human miRNA is transcribed into a precursor, a final mature miRNA may be formed by a cleavage.

miR-145-5p is microRNA-145-5p, a short RNA encoded by a MIR145 gene in humans. miR-145-5p may be present on human chromosome 5. Expression of miR-145-5p may be related to ovarian hormone secretion, activation of ovarian function, and ovarian reserve. Specifically, when the expression of miR-145-5p decreases, it may be that an ovarian reserve is high, the ovary is highly functional, or fertility is high.

miR4245-5p is microRNA-425-5p, a short RNA encoded by the MIR425 gene in humans. miR-425-5p may be present on human chromosome 3. Expression of miR-145-5p may be related to ovarian hormone secretion, activation of ovarian function, and ovarian reserve. Specifically, when the expression of miR-145-5p increases, it may indicate high ovarian reserve, highly functional ovary, or high fertility.

The gene may stimulate bone morphogenetic protein (BMP) signaling pathway. Specifically, miR-145-5p may target a gene belonging to the TGFβ superfamily. The gene may be at least one selected from the group consisting of Acvr1b, Acvr2a, Bmpr2, Tgfbr2, Smad1, and Smad3. In addition, the pathways of the TGFβ superfamily is largely divided into TGFβ signaling pathway and BMP signaling pathway, and miR-145-5p may target both of these pathways, and consequently stimulate the ovarian BMP signaling pathway.

When the BMP signaling pathway is stimulated, ovarian reserve may be enhanced through activation of dormant primordial follicles in the ovary, enhancement of ovarian hormones, and enhancement of ovarian function.

miR-425-5p may target Grem2, a BMP antagonist, and as a result, may stimulate the ovarian BMP signaling pathway.

The term “gene” refers to a nucleic acid sequence encoding a substance having a function, as a unit of genetic information. The gene may include an open reading frame (ORF). The gene may include regulatory sequences including promoters as well as ORFs.

Expression level of the gene may be the expression level of a transcript transcribed or translated from the gene and a protein or a fragment thereof translated therefrom. The transcript may include mRNA, non-coding RNA, or complementary DNA (cDNA) thereof. The fragment may be a part of the protein and may be an immunogenic polypeptide.

The term “expression level” refers to an amount of a protein or an amount of a transcript. The expression level may be a relative proportion of a protein or a transcript. For example, an increase in the expression level may be an increase in an amount of a protein or a transcript compared to a negative control group.

The agent may be an antibody or an antigen-binding fragment thereof that binds specifically to a protein expressed by the gene or a fragment thereof. The antibody may be a polyclonal antibody or a monoclonal antibody. The term “antibody” may be used interchangeably with the term “immunoglobulin”. The antibody may be a polyclonal antibody or a monoclonal antibody. The antibody may be a full-length antibody. The antigen-binding fragment refers to a polypeptide including an antigen-binding site. The antigen-binding fragment may be a single-domain antibody, Fab, Fab′, or scFv. The antibody or antigen-binding fragment may be attached to a solid support. The solid support may be, for example, a surface of a metal chip, a plate, or a well.

The agent may be a nucleic acid including a polynucleotide identical to or complementary to the nucleic acid sequence of the gene. The nucleic acid may be a primer or a probe. The primer or probe may be labeled with a fluorescent material, chemiluminescent material, or radioactive isotope at the end or inside.

The term “complementary” means that an antisense oligonucleotide is sufficiently complementary to selectively hybridize with the miRNA target under certain hybridization or annealing conditions, preferably physiological conditions, and the meaning may encompass both being in part or partially substantially complementary and perfectly complementary. “Substantially complementary” means being complementary enough to produce an effect according to the present specification, that is, to interfere with the activity of miRNA, although not perfectly complementary.

The term “nucleic acid” includes polynucleotides, oligonucleotides, DNA, RNA, and analogs and derivatives thereof, including, for example, peptide nucleic acids (PNA) or mixtures thereof. In addition, the nucleic acid may be single or double-stranded, and may encode a molecule including a polypeptide, mRNA, microRNA, or siRNA.

The term “ovarian reserve” refers to a marker indicating a number of oocytes in the ovary that may be ovulated, and quality of the oocytes obtained by induction of ovulation. The ovarian reserve may refer to an index indicating fertility, ovarian function, or early menopause.

In an example, the ovarian reserve may indicate whether or not there is activation of primordial follicles, production of ovarian hormones, or ovarian hypofunction.

The term “ovarian hypofunction” may refer to a condition in which a risk of developing a disease related to ovarian hypofunction is high due to ovarian hypofunction. Ovarian function may be identified by measuring changes in levels of follicle-stimulating hormone (FSH), estradiol, or anti-Mullerian hormone (AMH). In addition, ovarian function may be confirmed by using a diagnostic composition according to an aspect. The ovarian hypofunction as described above may cause hormonal imbalance, premature ovarian failure, polycystic ovary syndrome, infertility or early menopause.

Diseases related to ovarian hypofunction include at least one selected from the group consisting of premature ovarian failure, polycystic ovary syndrome, subfertility, premature menopause, oligomenorrhea, menstrual irregularity, ovarian failure, hyperandrogenism, anemia, amenorrhea, morphological polycystic, insulin resistance, and compensatory hyperinsulinemia.

The term “low ovarian reserve” may mean that a number of oocytes in the ovaries is small and the quality of the oocytes is relatively low. Therefore, low ovarian reserve may mean low fertility, a high risk of premature menopause, or a high risk of diseases related to ovarian hypofunction. In addition, low ovarian reserve may also mean a decrease in ovarian function.

“High ovarian reserve” may mean that a number of oocytes in the ovaries is large and the quality of the oocytes is relatively high. Thus, high ovarian reserve may mean high fertility, a low risk of early menopause or a low risk of diseases related to ovarian hypofunction. In addition, high ovarian reserve may also mean an increase in ovarian function.

The term “diagnosis” refers to determining a disease name, and may include determining an ovarian reserve, disease name, disease state, disease stage, etiology, presence or absence of complications, prognosis, and recurrence.

Another aspect provides a kit for diagnosing ovarian reserves, including an agent measuring expression levels of one or more genes selected from the group consisting of miR-145-5p and miR-425-5p.

Another aspect provides a kit for diagnosing ovarian dysfunction, or early menopause including an agent measuring expression levels of one or more genes selected from the group consisting of miR-145-5p and miR-425-5p.

The gene may be isolated from a biological sample isolated from a subject suspected of ovarian hypofunction or a subject suspected of having low ovarian reserve. The subject may be a mammal, and may be a human, a dog, a cat, a rat, a mouse, a hamster, a rabbit, a horse, sheep, a cow, a goat or a pig.

The biological sample may be a blood-derived sample or a cell-derived sample. For example, the biological sample may be plasma or blood cells.

The subject, biological sample, gene, expression level, agent, ovarian reserve, and diagnosis are as described above.

The kit may further include a sample required for diagnosis of ovarian reserve. The kit may include a solid support, a substrate for immunological detection of an antibody or antigen-binding fragment, an appropriate buffer, a chromogenic enzyme, a secondary antibody labeled with a fluorescent substance, or a chromogenic substrate. The kit may include a polymerase, a buffer, a nucleic acid, a coenzyme, a fluorescent material, or a combination thereof for nucleic acid detection. The polymerase may be, for example, a Taq polymerase.

Still another aspect provides a method of detecting a marker to provide information for diagnosing ovarian reserve, including: measuring an expression level of miR-145-5p or miR-425-5p in a subject suspected of having ovarian hypofunction; and comparing the measured expression level with an expression level of the gene of the normal control group.

Another aspect provides a method of detecting a marker to provide information for diagnosing ovarian dysfunction or early menopause, including: measuring an expression level of miR-145-5p or miR-425-5p in a subject suspected of having ovarian hypofunction; and comparing the measured expression level with an expression level of the gene of the normal control group.

The subject, biological sample, gene, expression level, agent, ovarian reserve, and diagnosis are as described above.

The gene may be isolated from a biological sample isolated from a subject suspected of ovarian hypofunction or a subject suspected of having low ovarian reserve. The subject may be a mammal, and may be a human, a dog, a cat, a rat, a mouse, a hamster, a rabbit, a horse, sheep, a cow, a goat or a pig.

The biological sample may be a sample derived from blood or a sample derived from cells. The blood-derived sample may be serum, plasma, and peripheral blood mononuclear cells (PBMC) separated from blood. The PBMC may include T-cells, B-cells, natural killer (NK) cells, monocytes, macrophages, dendritic cells, or a combination thereof. Preferably, the blood-derived sample may be serum or plasma. The sample may include transcripts or proteins.

The method may include determining that the subject has high ovarian reserve, a low risk of early menopause, or high ovarian function, when the measured expression level of the miR-145-5p gene is decreased compared to a measured expression level in the normal control group; or when the measured expression level of the miR-425-5p gene is increased compared to a measured expression level in the normal control group.

In addition, the method may further include determining that the subject has a low risk of premature ovarian failure, when the measured expression level of the miR-145-5p gene is decreased compared to a measured expression level in the normal control group; or when the measured expression level of the miR-425-5p gene is increased compared to a measured expression level in the normal control group.

The subject may be a mammal, for example, a human, a dog, a cat, a mouse, a rabbit, a horse, sheep, a hamster, a hedgehog, a ferret, or a guinea pig. The subject may be a subject suspected of having decreased ovarian reserve or ovarian dysfunction. Specifically, ovarian dysfunction may refer to all phenomena resulting in decreased fertility, including a decrease in ovarian hormones or a decrease in ovarian activity.

The blood-derived sample may be serum, plasma, or a combination thereof.

The measuring may include incubating the blood-derived sample with a nucleic acid including a polynucleotide identical to or complementary to the nucleic acid sequence of the gene.

The measuring may be performed by electrophoresis, immunoblotting, enzyme-linked immunosorbent assay (ELISA), immunohistochemical staining, protein chip, immunoprecipitation, microarray, northern blotting, polymerase chain reaction (PCR), reverse transcription-PCR (RT-PCT), real-time PCR, or a combination thereof.

The method includes comparing the measured expression level with an expression level of the gene of the normal control group.

The term “normal control” may be used interchangeably with the term “negative control”. The normal control group may be subjects that have never had a disease causing a decrease of ovarian reserve, or healthy subjects.

Another aspect provides a method of providing information for diagnosing ovarian dysfunction, infertility, or subfertility including: measuring an expression level of at least one gene selected from the group consisting of miR-145-5p and miR-425-5p, in a subject suspected of ovarian hypofunction or decrease of ovarian reserve; and comparing the measured expression level with an expression level of the gene of the normal control group.

The subject, biological sample, gene, expression level, agent, ovarian reserve, and diagnosis are as described above.

Another aspect provides a use of an agent capable of measuring expression levels of miR-145-5p or miR-425-5p, which is a biomarker for predicting prognosis of cancer.

### Advantageous Effects of Disclosure

According to a composition and a kit for diagnosing ovarian reserve, a method of diagnosing ovarian reserve using the same or a method of providing information for diagnosing ovarian reserve, according to an aspect, ovarian reserve may be diagnosed early. In addition, it is possible to predict not only the number of the oocytes but also the quality of the oocytes to make a comprehensive diagnosis of ovarian reserve.

### BRIEF DESCRIPTION OF DRAWINGS

FIG. 1 shows a schematic diagram of a plan of stem cell treatment for functional recovery of aged ovaries.

FIGS. 2A and 2B show images for analyzing implantability in the ovary of human placenta-derived mesenchymal stem cells (hPD-MSCs) injected into aged mouse models.

FIG. 3A shows a graph showing numbers of primary follicles after stem cell treatment; FIG. 3B is a graph showing E2 expression after stem cell treatment; and FIG. 3C is a graph showing changes of a marker of early follicle development after stem cell treatment.

FIGS. 4A and 4B show graphs and tables for analyzing circulating miRNAs related to early follicle development, based on miRNA-Seq.

FIG. 5A shows a table showing the target of miR-145-5p; FIG. 5B shows a graph identifying changes in protein expression due to a change in miRNA expression; FIG. 5C shows a table showing the miR-425-5p target; and FIG. 5D shows an image for analyzing an effect of selected miRNA on the bone morphogenetic protein (BMP) signaling pathway related to activation of primordial follicles.

FIG. 6 is a schematic diagram showing the principle by which changes in the expression of miR-145 and miR-425 by stem cell treatment stimulate the BMP signaling pathway and the activation of primordial follicles.

### MODE OF DISCLOSURE

Hereinafter, the present disclosure will be described in more detail through examples. However, these examples are intended to illustrate the present disclosure, and the scope of the present disclosure is not limited to these examples.

### Example 1. Selection and Identification of Biomarkers for Ovarian Reserve or Premature Ovarian Failure

### 1. Stem Cell Treatment for Restoring Function of Aged Ovaries

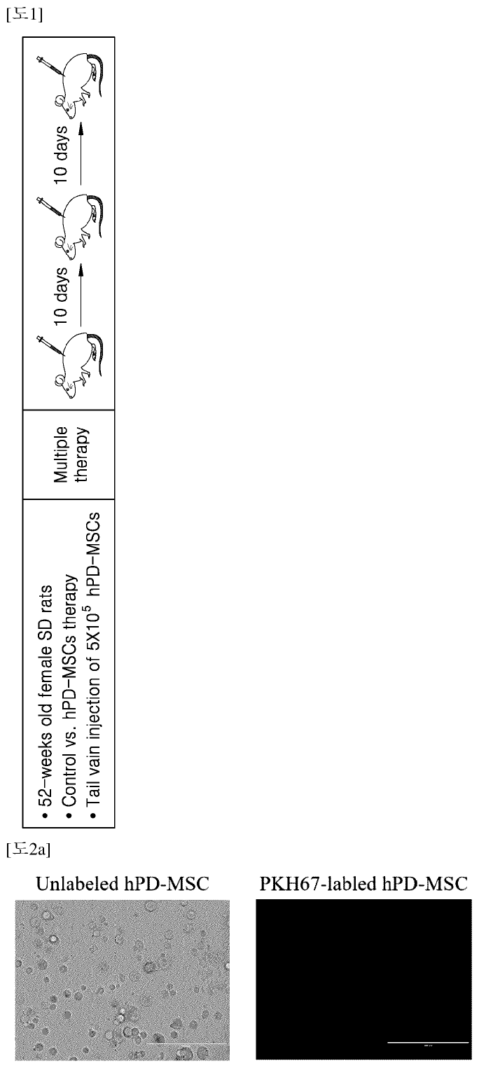

As shown in FIG. 1, 5×10⁵ human placenta-derived mesenchymal stem cells were injected through the tail vein of naturally aged 52-week-old female mice three times at 10-day intervals, and groups at week 1, week 2, week 3, and week 5 were defined as stem cell treatment experimental groups (hereinafter referred to as “experimental groups”), and a group to which the same amount of PBS was injected under the same conditions was defined as a control group, and the groups were analyzed for comparison.

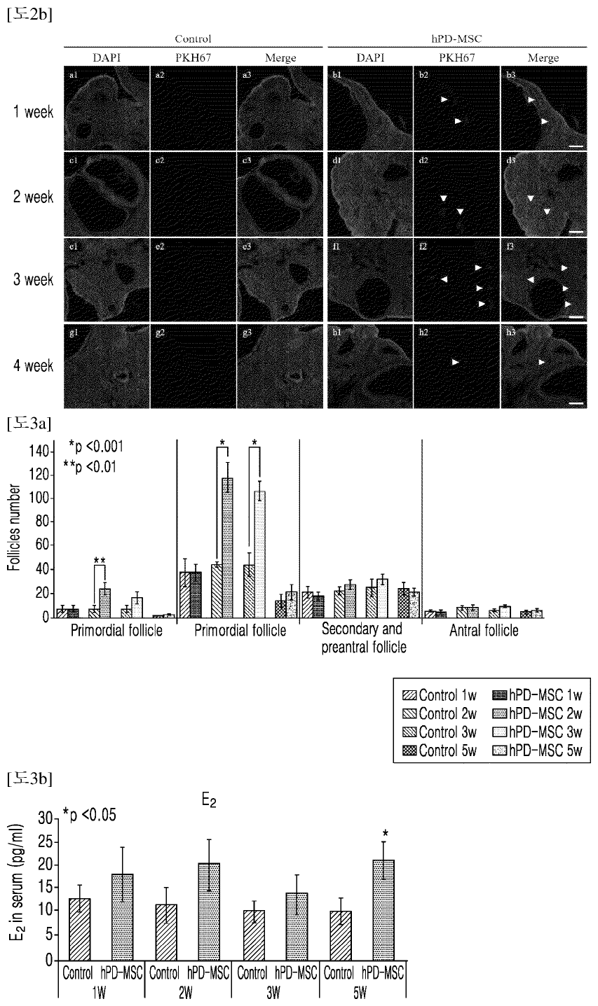

Human placenta-derived mesenchymal stem cells injected into aged mice were stained with PKH67 to confirm whether the stem cells were settled in the ovary, as shown in FIG. 2A. PKH67 staining was performed immediately before cell injection using PKH67 Green Fluorescent Cell Linker Kits (Sigma-Aldrich). The extracted ovaries were washed with Dulbecco's phosphate-buffered saline (DPBS) and then fixed with 4% paraformaldehyde. After making a frozen block at the optimal cutting temperature (OCT), frozen sections with a thickness of 12 μm were prepared. The prepared frozen sections were fixed and counter stained with 4′,6-diamidino-2-phenylindole (DAPI) and then, the presence of human placenta-derived mesenchymal stem cells in the aged ovary was identified by using a confocal microscope.

As a result, as shown in FIG. 2B, it was confirmed that the human placenta-derived mesenchymal stem cells stained with PKH67 were settled in the ovary from week 1 after injection, and were also observed to be settled around the follicle. Also, human placenta-derived mesenchymal stem cells could be observed in the samples of the mice of the experimental group at week 5 after injection.

The above results indicate that when stem cells are injected through the tail vein of aged mice three times at 10-day intervals, the injected cells are well settled in the ovaries.

Next, to analyze efficacy of the human placenta-derived mesenchymal stem cells settled in the aged ovary, the number of follicles and the levels of hormones in the blood were analyzed.

The ovaries, extracted to identify the number of follicles, were fixed by using 4% paraformaldehyde, and then paraffin blocks were prepared. The entire ovary was cut to a thickness of 7 μm to make paraffin sections. In this regard, the tissue sections were sequentially attached to polarized slides. The slides of the 10th slide number were selected and subjected to deparaffinization and fixation, followed by hematoxylin and eosin (H&E) staining. The stained slides were analyzed for the number of follicles according to the stage of follicle development (primordial follicle, primary follicle, secondary follicle, and antral follicle) using an optical microscope. Classification of follicles according to developmental stages is as follows. A primordial follicle is in a form in which several flattened granulosa cells surround an oocyte, a primary follicle is a follicle in which granulosa cells are transformed to cuboidal granulosa cells as the primordial follicle starts to grow. Secondary follicles are follicles into which primary follicles have grown, and are follicles in which granulosa cells have increased to two layers or more, while theca cells begin to appear outside the granulosa cells. The number of granulosa cells continues to increase, and when the cells grow beyond a certain level, an antrum is formed in the follicle, which is called an antral follicle.

E2 and AMH were measured in the serum. After fasting the mice for 12 hours, breathing anesthesia was performed with CO₂ gas, blood was collected from the abdominal aorta by using a syringe, and a serum separation tube (polyethylene tube) was used for various blood biochemical tests. Serum was separated from the collected blood using a centrifuge within 40 minutes after blood collection. For measurement of hormones, the serum was separated by 300 μl and stored at −20° C. E2 concentration in the serum was measured by using Elecsys® Estradiol III (Roche Diagnostics GmbH), and the AMH concentration was measured by using Elecsys® AMH immunoassay (Roche Diagnostics GmbH). Values of both hormones were measured by using the cobas 6000 system (Roche Diagnostics GmbH), and a concentration of each hormone in the serum was calculated by using the standard curve of the standard substance.

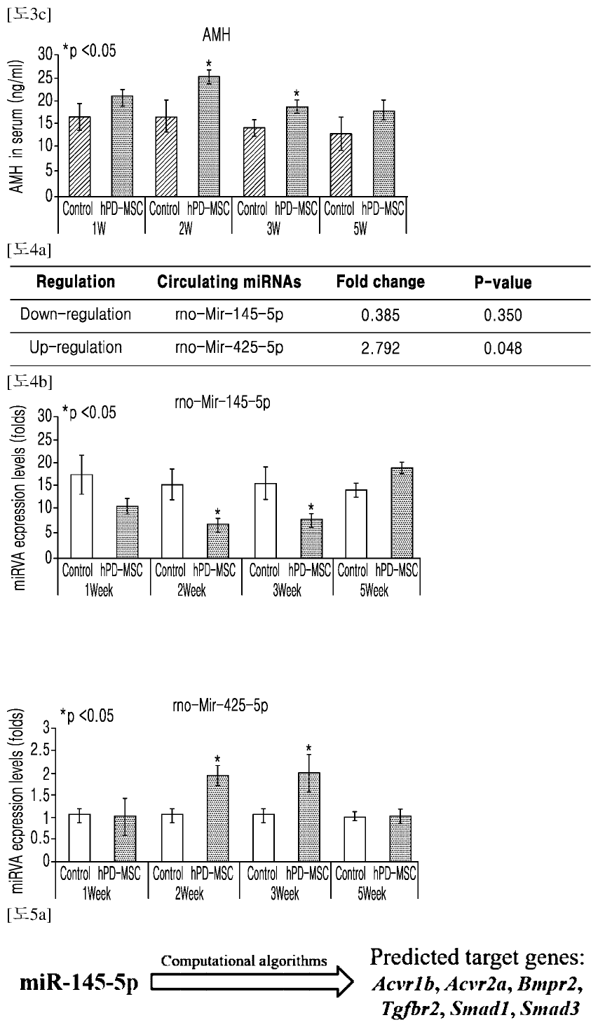

As a result, as shown in FIG. 3A, after repeated injection of human placenta-derived mesenchymal stem cells three times, it was observed that the number of primary follicles more than doubled in the week-2 and week-3 groups.

In addition, as a result of comparing E2 produced in the ovaries in the experimental group and the control group, as shown in FIG. 3B, E2 was observed to be higher in the blood of mice injected with stem cells. In addition, as shown in FIG. 3C, it was also observed that the concentration of AMH, which is a marker of early follicle development, was significantly increased in the week-2 and week-3 groups after stem cell injection.

The above results indicate that the human placenta-derived mesenchymal stem cells settled in the aged ovary activate the dormant primordial follicles. That is, when human placenta-derived mesenchymal stem cells settle in the aged ovary, the stem cells were confirmed to improve ovarian reserve by increasing development of early follicles, and a phenotype of enhanced ovarian function was observed through production of ovarian hormones.

### 2. Selection of Circulating miRNA Biomarkers

miRNA-Seq was performed, in order to select circulating miRNAs related to early follicle development. More specifically, based on the data of Example 1, it was confirmed that the number of primary follicles in the plasma of the mice group at week 2 after stem cell injection sharply increased, and based on this, the plasma of the mice in the experimental group at week 2 after injection was collected and miRNA-Seg was performed.

Circulating miRNAs were isolated from serum of the animals at week 2 after injection of human placenta-derived mesenchymal stem cells using a miRNeasy serum/plasma kit (Qiagen). eBiogen Co., Ltd. was requested to perform the miRNA-seq. Briefly, concentration of circulating miRNA was measured using a trace spectrophotometer (ND 2000; Nano Drop), and then adjusted to 200 ng, before applying the miRNA to the experiment. Library construction was performed by using a multiple small RNA library prep kit (NEB), and miRNA-seq was performed by using IIlumina NextSeq500 (IIlumina) and Illumina SE75 (IIlumina). Thereafter, using Excel-based miRNA-seq data analysis (ExDEGA) tool provided by eBiogen, circulating miRNAs which are expressed significantly (p<0.05) differently by two-fold or more compared to the control group were selected.

Circulating miRNA was isolated from 200 μl of serum by using an miRNeasy serum/plasma kit (Qiagen), and during the isolation process, cel-miR-39 mimic (1.6×10⁸ copies/μl; Qiagen) was added and used as an external spike-in control. The extracted circulating miRNA was reverse transcribed using a HB_I RT Reaction kit (Heimbiotek). All quantitative real-time RT-PCR analyses were performed by using a CFX96 Touch Real-Time PCR Detection System (Bio-Rad) and a Nucleic mix II kit. The PCR reactant was made by using HB miR Multi assay kit system I, and PCR was performed under the reaction cycle conditions of 15 minutes at 95° C., followed by 40 cycles of 10 seconds at 95° C., and single fluorescence measurement for 40 seconds at 60° C. The primers used in the experiment were purchased from Heimbiotek, and the measured miRNA expression level was corrected by the expression level of cel-miR-39.

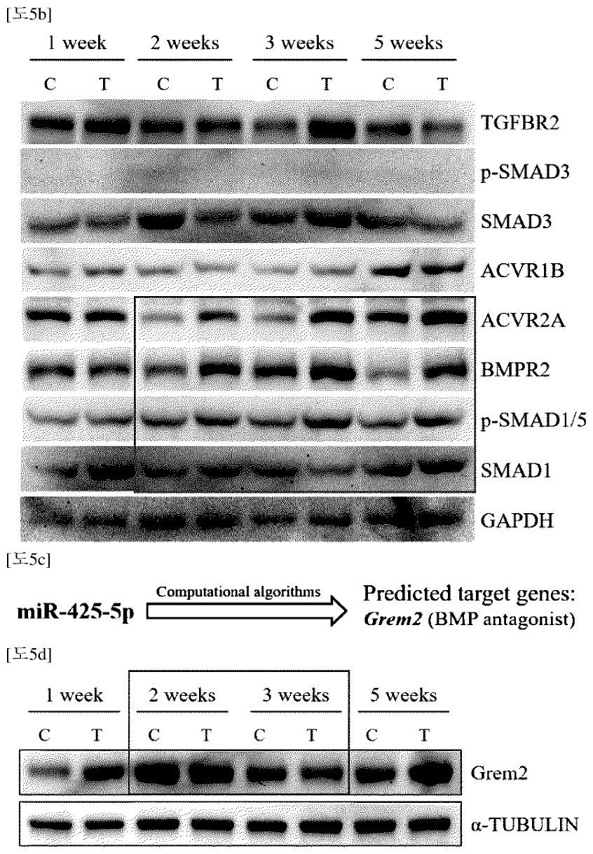

As a result, as shown in FIG. 4A, various circulating miRNAs that changed after stem cell injection into aged mice could be identified, and among them, underexpressed miR-145-5p and overexpressed miR-425-5p compared to the control group were selected in the serum of the groups injected with stem cells.

Next, quantitative real-time RT-PCR was performed to verify expression of the selected circulating miRNA. As a result, as shown in FIG. 4B, it was confirmed that expression of miRNA-145-5p was decreased at week 2 and week 3 after injection of human placenta-derived mesenchymal stem cells, and expression of miRNA-425-5p was increased.

### 3. Confirmation of Stimulation of BMP Signaling Pathway of Selected Biomarkers

In order to confirm the subgenes of the circulating miRNA selected in Example 1 above, the results were analyzed using various methods of bioinformatics (computational algorithms) such as miRWalk, TargetScan, miRmap, and miRanda.

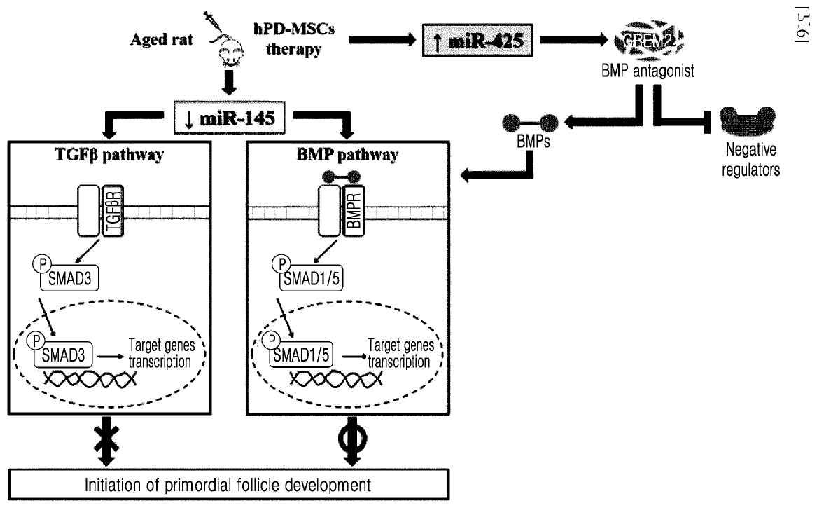

As a result, as shown in FIG. 5A, miR-145-5p was found to target genes belonging to the TGFβ superfamily (Acvr1b, Acvr2a, Bmpr2, Tgfbr2, Smad1, Smad3). The pathways of the TGFβ superfamily is largely divided into the TGFβ signaling pathway and the BMP signaling pathway, and it was confirmed that the miR-145-5p targets both of these pathways.

Changes in the expression of sub-proteins due to changes in the expression of the selected circulating miRNAs were confirmed in the ovarian tissue by using Western blotting. Proteins were extracted from the ovarian tissue using a lysis buffer and then electrophoresed on 10% or 12% SDS-polyacrylamide gel. The extracted proteins were transferred to a polyvinyldene difluoride membrane (PVDF membrane), and then reacted with a TBS-T buffer solution containing 5% skim milk for 1 hour. After reacting the proteins with primary antibodies overnight at 4° C., secondary antibodies attached to horseradish peroxidases (HRP) were reacted with the proteins for 1 hour at room temperature, and then protein expression was confirmed using the enhanced chemiluminescence (ECL) method.

As shown in FIG. 5B, as a result of confirming change of the protein expression by Western blot, in the ovaries of the groups at week 2, week 3, and week 5 after stem cell injection that underexpress miR-145-5p, expression of proteins related to the BMP signaling pathway (Acvr2a, Bmpr2, Smad1, p-Smad1/5) was confirmed to be higher than that of the control group.

In addition, as shown in FIG. 5D, it was confirmed that miR-425-5p targets Grem2, a BMP antagonist, and as a result of confirming the change in protein expression in the ovaries, it was observed that protein expression was lower in the week-2 and week-3 groups after stem cell injection compared to the control group.

The above results indicate that the expression of circulating miRNAs present in the blood is changed by human placenta-derived mesenchymal stem cells injected into aged mice, and the change in their expression stimulates the BMP signaling pathway in the ovary.

As a result, as shown in FIG. 6, the stimulation of the BMP pathway suggests that the dormant primordial follicles in the ovary are activated due to the activation of the BMP signaling pathway in the ovary. When follicle development from the stage of the primordial follicle to the primary follicle resumed, the follicle growth proceeded vigorously, and as a result, production and secretion of ovarian hormones also increased, suggesting that the ovarian function of the aged mice was improved.

Therefore, changes in the expression of miR-145-5p and miR-425-5p present in the blood may be used as a marker of follicle development to predict ovarian reserve of women. In addition, changes in the expression of miR-145-5p and miR-425-5p present in the blood may predict ovarian function, such as hormone production, and be used as a marker for diagnosis of early ovarian failure.

1. A composition for diagnosing ovarian reserve, comprising an agent capable of measuring expression levels of miR-145-5p or miR-425-5p.

2. The composition of claim 1, wherein the ovarian reserve indicates whether or not there is activation of primordial follicles, production of ovarian hormones, or ovarian hypofunction.

3. The composition of claim 1, wherein the agent is a nucleic acid comprising a polynucleotide identical to or complementary to the nucleic acid sequence of the miRNA.

4. The composition of claim 3, wherein the nucleic acid is a primer or a probe.

5. The composition of claim 1, wherein the miRNA stimulates a bone morphogenetic protein (BMP) signaling pathway.

6. A kit for diagnosing ovarian reserve, comprising an agent capable of measuring expression levels of miR-145-5p or miR-425-5p.

7. A method of detecting a marker to provide information for diagnosing ovarian reserve, comprising: measuring an expression level of miR-145-5p or miR-425-5p in a subject suspected of having ovarian hypofunction; and comparing the measured expression level with an expression level of a gene of a normal control group.

8. The method of claim 7, wherein the ovarian reserve indicates whether or not there is activation of primordial follicles, production of ovarian hormones, or ovarian hypofunction.

9. The method of claim 7, wherein the subject is a human, a dog, a cat, a mouse, a rat, a rabbit, a horse, sheep, a cow, a goat, or a pig.

10. The method of claim 7, wherein the measuring comprises incubating a nucleic acid comprising a polynucleotide identical to or complementary to the nucleic acid sequence of the gene.

11. The method of claim 7, wherein the measuring is performed by electrophoresis, immunoblotting, enzyme-linked immunosorbent assay (ELISA), immunohistochemical staining, protein chip, immunoprecipitation, microarray, northern blotting, polymerase chain reaction (PCR), reverse transcription-PCR (RT-PCT), real-time PCR, or a combination thereof.

12. The method of claim 8, further comprising, determining that the subject has high ovarian reserve or high activation of ovarian function, when the measured expression level of the miR-145-5p gene is decreased compared to the measured expression level in the normal control group; or when the measured expression level of the miR-425-5p gene is increased compared to the measured expression level in the normal control group.

13. The method of claim 8, further comprising, determining that the subject has a low risk of early ovarian failure, when the measured expression level of the miR-145-5p gene is decreased compared to the measured expression level in the normal control group, or when the measured expression level of the miR-425-5p gene is increased compared to the measured expression level in the normal control group.
